(19) United States
(12) Patent Application Publication
(10) Pub. No.: US 2022/0151896 A1
(43) Pub. Date: May 19, 2022

(54) EFFICIENT SUNSCREEN COMPOSITIONS WITH DIETHYLAMINO HYDROXYBENZOYL HEXYL BENZOATE AND ORGANIC PARTICULATE UV FILTER

(71) Applicant: BASF SE, Ludwigshafen am Rhein (DE)

(72) Inventors: Marcel SCHNYDER, Grenzach-Wyhlen (DE); Stanislaw KRUS, Grenzach-Wyhlen (DE); Stephannie ACKER, Grenzach-Wyhlen (DE); Myriam SOHN, Grenzach-Wyhlen (DE)

(21) Appl. No.: 17/439,581

(22) PCT Filed: Mar. 13, 2020
(86) PCT No.: PCT/EP2020/056942

§ 371 Date: Sep. 15, 2021

(30) Foreign Application Priority Data
Mar. 15, 2019 (EP) 19163167.0

(51) Int. Cl. A61K 8/41; A61K 8/49; A61Q 17/04

(52) U.S. Cl. CPC A61K 8/415; A61Q 17/04; A61K 8/4966

(57) ABSTRACT

The present invention relates to sunscreen or daily care compositions comprising diethylamino hydroxybenzoyl hexyl benzoate, at least one organic particulate UV filter and at least one UV filter selected from triazine derivatives, wherein the composition does not comprise octocrylene. Furthermore, the present invention relates to the use of said composition to reduce fabric staining and to facilitate the washability of a sunscreen or daily care composition.

The present invention relates to sunscreen or daily care compositions comprising diethylamino hydroxybenzoyl hexyl benzoate, at least one organic particulate UV filter and at least one UV filter selected from triazine derivatives, wherein the composition does not comprise octocrylene. Furthermore, the present invention relates to the use of said composition to reduce fabric staining and to facilitate the washability of a sunscreen or daily care composition.

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filter, UV-B filter and broadband filters. These filters are added to sunscreen or cosmetic compositions. The UV filters are either organic or inorganic, particulate or non-particulate compounds, of which all have a high absorption efficacy in the UV-light range. In general, UV light can be divided into UV-A radiation and UV-B radiation. Depending on the position of the absorption maxima, UV-filters are divided into UV-A and UV-B filters. In case an UV-filter absorbs both, UV-A and UV-B light, it is referred to as a broadband absorber.

Since 2006, the EU commission has recommended that all sunscreen or cosmetic compositions should have an UV-A protection factor, which is at least one third of the labelled sun protection factor (SPF), wherein the sun protection factor refers mainly to the UV-B protection.

However, the UV filters known in the prior art, which are used in sunscreen or cosmetic compositions have certain disadvantages. In particular, it is referred to the disadvantage of certain UV filter, which are frequently under discussion due to their health and environmental concern, although they are approved for being used in sunscreen or cosmetic compositions. UV filter under discussion are for example octocrylene, homosalate or ethylhexyl methoxy cinnamate.

Therefore, there is a need for sunscreen or cosmetic compositions of the daily use, which are efficient for sun protection in the UV-B and UV-A range but free of UV filter under discussion. EP 2 837 407 A2 discloses odor stable, octocrylene free cosmetic compositions comprising one or more UV filter selected from triazine derivatives, titanium dioxide, butyl methoxydibenzoylmethane and/or diethylamino hydroxybenzoyl hexyl benzoate, wherein the composition is free of ethylhexyl salicylate, octocrylene, homomenthylsalicylate and ethylhexyl methoxy cinnamate.

EP 3 093 008 A1 refers to a sunscreen composition which is free of octocrylene and comprises diethylamino hydroxybenzoyl hexyl benzoate, phenylbenzimidazole sulfonic acid, and one or more salicylates selected from ethylhexyl salicylate and homosalate.

EP 3 093 007 A1 discloses a cosmetic composition comprising a UV filter combination of diethylamino hydroxybenzoyl hexyl benzoate, ethylhexyl triazone and one or more salicylates selected from ethylhexyl salicylate and homosalate, wherein the composition may further be free of oxybenzone, octocrylene and 4-methylbenzylidene camphor.

EP 3 093 006 relates to an alcohol containing, octocrylene free sunscreen composition. The cosmetic composition comprises a UV filter combination of diethylamino hydroxybenzoyl hexyl benzoate, ethylhexyl triazone, and bis-ethylhexylphenol methoxyphenyl triazine, wherein the composition comprises one or more alcohols.

EP 3 195 853 A1 discloses an octocrylene free sunscreen composition, wherein the cosmetic composition comprises a UV filter combination of diethylamino hydroxybenzoyl hexyl benzoate, diethyl butamido triazone and ethylhexyl triazone.

EP 3 351 236 A1 relates to a cosmetic composition comprising a UV filter combination of diethylamino hydroxybenzoyl hexyl benzoate, butyl methoxydibenzoylmethane and polyglycerol fatty acid esters, wherein the composition is free of 4-methylbenzylidene camphor, oxybenzone, ethylhexyl methoxycinnamate, isoamyl p-methoxycinnamate and octocrylene.

EP 3 354 253 A2 describes the use of diethylamino hydroxybenzoyl hexyl benzoate in cosmetic preparations to reduce fabric staining.

US 2003/0161793 A1 discloses a topically applicable cosmetic/dermatological sunscreen composition devoid of any p-methylbenzylidene camphor but contains at least one UV-screening dibenzoylmethane compound and at least one UV-screening amino-substituted 2-hydroxybenzophenone compound of the structural formula (I).

US 2003/0152532 A1 relates to topically applicable cosmetic/dermatological sunscreen compositions comprising particulates of at least one insoluble organic UV-screening agent and at least one UV screening amino-substituted 2-hydroxybenzophenone compound.

Therefore, it has been an object of the present invention to provide efficient sunscreen or daily care compositions. It has been another object of the present invention to provide efficient sunscreen or daily care compositions, which perfectly meet the consumers' demands with respect to performance and convenience after application of the product. In this connection, it has been another object of the present invention to provide sunscreen or daily care compositions, which are free of UV filters under discussion. It has been another object of the present invention to provide compositions, which are suitable for use in sunscreen or daily care compositions in order to reduce fabric staining and facilitate the washability of textiles. It has surprisingly been found that at least one of these objects can be achieved by the sunscreen or daily care composition according to the present invention.

In particular, the inventors of the present application found that the sunscreen or daily care composition according to the present invention provides an efficient UV-A and UV-B protection, nevertheless the composition is free of certain UV filter under discussion, i.e. octocrylene. Furthermore, it has surprisingly been found by the inventors of the present application that the sunscreen or daily care composition according to the present invention can be used to reduce fabric staining and facilitate the washability of the sunscreen or daily care composition from textiles.

Thus, according to one embodiment, the present invention relates to a sunscreen or daily care composition comprising
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives;
   wherein the composition does not comprise ethylhexyl-2-cyano-3,3-diphenyl-acrylate (INCI octocrylene).

In a second embodiment, the present invention relates to a sunscreen or daily care composition, in the form of an emulsion comprising
(i) hexyl 2-[4-(diethylamino)-2-hydroxybenzoyl]benzoate (INCI diethylamino hydroxybenzoyl hexyl benzoate) in an amount of from 2.5% to 9% by weight, based on the total weight of the composition; and
(ii) at least one organic particulate UV filter; and
(iii) at least one UV filter selected from triazine derivatives selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone) and combinations thereof;
   wherein the composition does not comprise ethylhexyl-2-cyano-3,3-diphenyl-acrylate (INCI octocrylene).

In a preferred embodiment of said composition according to the first and second embodiment, the sunscreen or daily care composition does not comprise 2-ethylhexyl-(2E)-3-(4-methoxyphenyl)acrylate (INCI ethylhexyl methoxy cinnamate).

In another preferred embodiment of said composition according to the first and second embodiment, the sunscreen or daily care composition is free of parabens.

In another preferred embodiment of said composition according to the first embodiment, the at least one UV filter selected from triazine derivatives is selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone), 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine) and combinations thereof.

In another preferred embodiment of said composition according to the first and second embodiment, the at least one organic particulate UV filter is selected from the group consisting of 2,2′-methylene bis[6-(2H-benzotriazol-2-yl)-4-(1,1,3,3-tetramethylbutyl)phenol] (INCI methylene bis-benzotriazolyl tetramethylbutylphenol), 2,4,6-tris(biphenyl-4-yl)-1,3,5-triazine (INCI trisbiphenyl triazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine), 5,6,5′,6′-tetraphenyl-3-3′-(1,4-phenylene)bis(1,2,4-triazine) (INCI phenylene bis-diphenyltriazine), micronized 1,4-di(benzoxazole-2′-yl)benzene and combinations thereof.

In another preferred embodiment of said composition according to the first and second embodiment, the composition further comprises at least one emollient with a polarity index of <30 mN/m.

In a more preferred embodiment of said composition according to the first and second embodiment, the at least one emollient is selected from the group consisting of C₁₂-C₁₅ alkyl benzoate, caprylic/capric triglyceride, butylene glycol dicaprylate/dicaprate, propylene glycol dicaprylate/dicaprate, diisopropyl sebacate, octyldodecanol, isopropyl palmitate, isopropyl myristate, dicaprylyl carbonate, phenethyl benzoate, dibutyl adipate, diisopropyl adipate, triethyl citrate and tributyl citrate.

In another preferred embodiment of said composition according to the first and second embodiment, the sunscreen or daily care composition comprises hexyl 2-[4-(diethylamino)-2-hydroxybenzoyl]benzoate (INCI diethylamino hydroxybenzoyl hexyl benzoate) in an amount of from 3% to 8% by weight, preferably in an amount of from 4% to 7% by weight based on the total weight of the composition.

In another preferred embodiment of said composition according to the first and second embodiment, the sunscreen or daily care composition comprises the at least one organic particulate UV filter in an amount of from 0.5% to 10% by weight, preferably in an amount of from 1% to 5% by weight, based on the total weight of the composition.

In another preferred embodiment of said composition according to the first and second embodiment, the sunscreen or daily care composition comprises the at least one UV filter selected from triazine derivatives in an amount of from 0.5% to 5% by weight, preferably in an amount of from 0.8% to 3% by weight, based on the total weight of the composition.

In another preferred embodiment of said composition according to the first and second embodiment, the sunscreen or daily care composition comprises at least one perfume.

In another preferred embodiment of said composition according to the first and second embodiment, the sunscreen or daily care composition is free of phenoxyethanol.

In another preferred embodiment of said composition according to the first and second embodiment, the sunscreen or daily care composition has an UV-A protection, which is at least one third of the sun protection factor of the sunscreen composition.

In another aspect the present invention relates to the use of a sunscreen or daily care composition, in the form of an emulsion, to reduce fabric staining, wherein the composition comprises
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives; and
   wherein the composition does not comprise ethylhexyl-2-cyano-3,3-diphenyl-acrylate (INCI octocrylene).

In a third aspect the present invention relates to the use of a sunscreen or daily care composition, in the form of an emulsion, to facilitate the washability from textiles, wherein the composition comprises
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives; and
   wherein the composition does not comprise ethylhexyl-2-cyano-3,3-diphenyl-acrylate (INCI octocrylene).

In a preferred embodiment of said uses, the sunscreen or daily care composition does not comprise 2-ethylhexyl-(2E)-3-(4-methoxyphenyl)acrylate (INCI ethylhexyl methoxy cinnamate).

In another preferred embodiment of said uses, the sunscreen or daily care composition is free of parabens.

In another preferred embodiment of said uses, the at least one UV filter selected from triazine derivatives is selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone), 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine) and combinations thereof.

In another preferred embodiment of said uses, the at least one organic particulate UV filter is selected from the group consisting of 2,2′-methylene bis[6-(2H-benzotriazol-2-yl)-4-(1,1,3,3-tetramethylbutyl)phenol] (INCI methylene bis-benzotriazolyl tetramethylbutylphenol), 2,4,6-tris(biphenyl-4-yl)-1,3,5-triazine (INCI trisbiphenyl triazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine), 5,6,5′,6′-tetraphenyl-3-3′-(1,4-phenylene)bis(1,2,4-triazine) (INCI phenylene bis-diphenyltriazine), micronized 1,4-di(benzoxazole-2′-yl)benzene and combinations thereof.

In another preferred embodiment of said uses, the composition further comprises at least one emollient with a polarity index of <30 mN/m.

In a more preferred embodiment of said uses, the at least one emollient is selected from the group consisting of C₁₂-C₁₅ alkyl benzoate, caprylic/capric triglyceride, butylene glycol dicaprylate/dicaprate, propylene glycol dicaprylate/dicaprate, diisopropyl sebacate, octyldodecanol, isopropyl palmitate, isopropyl myristate, dicaprylyl carbonate, phenethyl benzoate, dibutyl adipate, diisopropyl adipate, triethyl citrate and tributyl citrate.

In another preferred embodiment of said uses, the composition comprises hexyl 2-[4-(diethylamino)-2-hydroxybenzoyl]benzoate (INCI diethylamino hydroxybenzoyl hexyl benzoate) in an amount of from 0.5% to 10% by weight, based on the total weight of the composition.

In another preferred embodiment of said uses, the composition comprises the at least one organic particulate UV filter in an amount of from 0.5% to 10% by weight, preferably in an amount of from 1% to 5% by weight, based on the total weight of the composition.

In another preferred embodiment of said uses, the composition comprises the at least one UV filter selected from triazine derivatives in an amount of from 0.5% to 5% by weight, preferably in an amount of from 0.8% to 3% by weight, based on the total weight of the composition.

In another preferred embodiment of said uses, the sunscreen or daily care composition comprises at least one perfume.

In another preferred embodiment of said uses, the sunscreen or daily care composition is free of phenoxyethanol.

In another preferred embodiment of said uses, the sunscreen or daily care composition has an UV-A protection, which is at least one third of the sun protection factor of the sunscreen composition.

In another aspect, the present invention relates to a sunscreen composition comprising
(i) diethylamino hydroxybenzoyl hexyl benzoate; and
(ii) bis-ethylhexyloxyphenol methoxyphenyl triazine; and
(iii) ethylhexyl triazone; and
(iv) methylene bis-benzotriazolyl tetramethylbutylphenol and/or trisbiphenyl triazine,
   wherein the sunscreen composition has an in vitro Boots rating UVA:UVB ratio of at least 0.9 before and 0.86 after an UV irradiation of 17.5 J/cm².

Before describing preferred embodiments of the present invention in detail, definitions important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention, the term “consisting of” is considered to be a preferred embodiment of the term “comprising of”. If hereinafter a group is defined to comprise at least a certain number of embodiments, this is meant to also encompass a group, which preferably consists of these embodiments only.

Furthermore, the terms “first”, “second”, “third” or “(i)”, “(ii)”, “(iii)”, “(iv)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

As used herein the term “free of” in the context that the composition of the present invention is free of a specific compound or group of compounds, which may be combined under a collective term, means that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all. The same definition is applied for the term “does not comprise”.

The term “sunscreen composition” refers to any topical product, which reflects and/or absorbs certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, or gels. The sunscreen composition may comprise one or more active agents, e.g., organic or inorganic UV filters, as well as other ingredients or additives, e.g., emulsifiers, emollients, viscosity regulators, stabilizers, preservatives, or fragrances.

The term “daily care composition” refers to any topical product, which reflects and/or absorbs certain parts of UV radiation and is used as an everyday care product for the human body, e.g., for face, body or hair. The daily care composition may comprise one or more active agents, e.g., organic or inorganic UV filters, as well as other ingredients or additives, e.g., emulsifiers, emollients, viscosity regulators, stabilizers, preservatives, or fragrances.

The term “emulsion” refers to a composition, in particular to a sunscreen or daily care composition, which is present in the form of an emulsion. Thus, the term “emulsion” refers to a mixture of two or more liquids that are normally immiscible. Emulsions represent a two-phase system, wherein one liquid (the dispersed phase) is dispersed in the other liquid (the continuous phase). Examples of preferred emulsions of the present application are W/O, O/W, O/W/O, W/O/W or PIT emulsions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The term “UV-filter” as used herein refers to organic or inorganic compounds, which can absorb and/or reflect UV radiation caused by sunlight. UV-filter can be classified based on their UV protection curve as UV-A, UV-B or broadband filters. In the context of the present application, broadband filters may be listed as UV-A filters, as they also provide UV-A protection. In other words, preferred UV-A filters also include broadband filters. Particulate UV filters can be further divided into organic particulate UV filters and inorganic particulate UV filters. While organic particulate UV filters are based on organic compounds, inorganic particulate UV filters are based on inorganic compounds such as titanium dioxide. In the sunscreen composition, particulate UV filters will be present in particulate form, as their solubility is less than 0.01% by weight, preferably less than 0.05% by weight in the sunscreen composition, i.e. in the water and the cosmetic oils contained therein. Preferably, the particulate UV filters have a particle size D_{N}50 determined by light scattering of less than 2000 nm, preferably less than 1000 nm, wherein D_{N}50 refers to the particle size value, where half of the population lies below this value, and half of the population lies above this value, i.e. the median value of the particle size volume distribution.

Hexyl 2-[4-(diethylamino)-2-hydroxybenzoyl]benzoate (INCI diethylamino hydroxybenzoyl hexyl benzoate) is an oil soluble UV-A filter, which has an absorption maximum at 354 nm. Diethylamino hydroxybenzoyl hexyl benzoate has an excellent photo stability for long-lasting protection of the skin and provides an efficient shielding against UV-A I and UV-A II radiation. It is sold under the trade name Uvinul® A Plus by BASF.

4,4′,4″-(1,3,5-triazine-2,4,6-triyltriimino)tris-benzoic acid-tris(2-ethylhexyl)ester (INCI ethylhexyl triazone) is an UV-B absorber with a high efficiency also at low concentrations. It is a highly stable UV-B filter. Ethylhexyl triazine is sold under the trade name Uvinul® T 150 by BASF.

4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone) is a soluble organic UV-B filter, which absorbs in the range of from 280 nm to 320 nm. It has an absorption maximum at 310 nm. Diethylhexyl butamidotriazone is sold by 3V Sigma under the trade name Uvasorb HEB.

2,2′-methylene bis[6-(2H-benzotriazol-2-yl)-4-(1,1,3,3-tetramethylbutyl)phenol] (INCI methylene bis-benzotriazolyl tetramethylbutylphenol) is a broadband UV filter, which absorbs UV-B light as well as UV-A light. Furthermore, it is a hybrid UV filter, which absorbs UV light as an organic compound, but also scatters some of the light, since it is a particulate UV filter, which is typically present in the form of micro fine organic particles. The particle size D_{N}50 determined by light scattering is preferably higher than 60 nm and the D_{N}90 is preferably higher than 110 nm. Methylene-bis-benzotriazolyl tetramethylbutylphenol (also referred to as MBBT) is a UV filter belonging to the group of benzotriazole derivatives. It is sold under the tradename Tinosorb® M by BASF.

2,4,6-Tris(biphenyl-4-yl)-1,3,5-triazine (INCI tris-biphenyl triazine), also referred to as TBPT, is a particulate organic UV filter belonging to the group of triazine derivatives. It is sold under the tradename Tinosorb® A2B by BASF. It provides protection in the UV-All and UV-B range with an absorption maximum at 310 nm. Preferably, the particles exhibit a D_{N}50 higher than 80 nm, and a D_{N}90 higher than 120 nm.

2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), also referred to as BEMT is an UV filter belonging to the group of triazine derivatives. It is sold under the tradename Tinosorb S by BASF. It is a broad spectrum UV filter. BEMT can be either provided as an oil soluble UV filter or dissolved in a polyacrylate matrix to be dispersible in water. In the latter case, it is referred to as Tinosorb® S Aqua or Tinosorb S Lite Aqua.

The term “dispersible in” as used herein indicates that a certain compound is not soluble in the respective water or oil phase, but finely dispersed in the respective phase.

The term “polymer matrix” as used herein is directed to matrices, which consist of multiple polymer chains grouped in a way to entrap, enclose or also dissolve molecules, such as different organic compounds.

Ethylhexyl-2-cyano-3,3-diphenyl-acrylate (INCI octocrylene) is an oil soluble organic UV-B filter with an absorption maximum at 302 nm. It provides a broad UV-B absorbance and is known to be an efficient stabilizer for photo-unstable UV filters. Octocrylene is sold by BASF under the tradename Uvinul® N 539 T.

2-Ethylhexyl-(2E)-3-(4-methoxyphenyl)acrylate (INCI ethylhexyl methoxy cinnamate) is an odorless and colorless UV-B filter with an absorption maximum at 310 nm. It is a good solvent for other crystalline UV filters. Ethylhexyl methoxy cinnamate is sold under the tradename Uvinul® MC 80 by BASF.

The term “paraben” refers to a class of preservatives used in cosmetic and pharmaceutical compositions. They are commonly used due to their bactericidal and fungicidal properties. The chemical structure of parabens is based on parahydroxybenzoates or esters of parahydroxybenzoic acid, e.g. methylparaben, ethylparaben, propylparaben, butylparaben or heptylparaben.

The term “emollient” relates to cosmetic preparations used for protecting, moisturizing and lubricating the skin. The word emollient is derived from the Latin word mollire, to soften. In general, emollients prevent evaporation of water from the skin by forming an occlusive coating. They can be divided into different groups depending on their polarity index.

The term “polarity index” refers to non-polar or polar oils. Non-polar oils are mainly based on hydrocarbons and lack an electronegative element, such as oxygen. In contrast, polar oils contain heteroatoms that differ in electronegativity, which results in a dipole moment. However, such oils are still insoluble in water, i.e. hydrophob. The polarity index can be determined by measuring the interfacial tension between the respective oil and water.

The term “C₁₂-C₁₅ alkyl benzoate” refers to esters of benzoic acid with fatty alcohols containing a C₁₂-C₁₅-alkyl chain. C₁₂-C₁₅ alkyl chain is defined as an alkyl chain with C₁₂, C₁₃, C₁₄ or C₁₅ chain length.

The definition of “broadband” protection (also referred to as broad-spectrum or broad protection) is based on the “critical wavelength”. For broadband coverage, UV—B and UV-A protection must be provided. According to the US requirements, a critical wavelength of at least 370 nm is required for achieving broad spectrum protection. Furthermore, it is recommended by the European Commission that all sunscreen or cosmetic compositions should have an UVA protection factor, which is at least one third of the labelled sun protection factor (SPF), e.g. if the sunscreen composition has an SPF of 30 the UVA protection factor has to be at least 10.

The term “fabric staining” as used herein refers to the observation that sunscreen compositions, in particular those providing good UV-A protection, leave yellow stains on textiles. This is undesired because the sunscreen composition is often in contact with textiles during or after the application to the human skin, so that stains will be observed on the textiles. Staining of fabrics can be measured by the L*a*b as outlined in detail below.

The term “washability” as used herein describes to which extent stains on textiles caused by sunscreen compositions can be removed from the textile after a certain number of washing cycles. In order to assess the washability, fabric staining is measured before and after one or more washing cycles. A washing cycle is carried out at a temperature of from 30° C. to 50° C. with a commercially available liquid washing gel. Typically, the difference regarding fabric staining after 1 to 3 washing cycles is determined.

Preferred embodiments regarding the sunscreen or daily care composition according to the present application, in particular according to the first and second embodiment, as well as the uses of said composition are described hereinafter. It is to be understood that the preferred embodiments of the invention are preferred alone or in combination with each other.

As indicated above, the present invention relates in one embodiment to a sunscreen or daily care composition, comprising
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives;
   wherein the composition does not comprise ethylhexyl-2-cyano-3,3-diphenyl-acrylate (INCI octocrylene).

As indicated above, the present invention relates in a second embodiment to a sunscreen or daily care composition in the form of an emulsion, comprising
(i) hexyl 2-[4-(diethylamino)-2-hydroxybenzoyl]benzoate (INCI diethylamino hydroxybenzoyl hexyl benzoate) in an amount of from 2.5% to 9% by weight, based on the total weight of the composition; and
(ii) at least one organic particulate UV filter; and
(iii) at least one UV filter selected from triazine derivatives selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone) and combinations thereof; wherein the composition does not comprise ethylhexyl-2-cyano-3,3-diphenyl-acrylate (INCI octocrylene).

In connection with the above embodiments, it has surprisingly been found by the inventors of the present application that the sunscreen or daily care composition as defined above provides an efficient sun protection, although the composition is free of the UV filter octocrylene.

In connection with the present invention, in particular in connection with the first and second embodiment, the following preferences regarding the sunscreen composition are relevant.

In one embodiment of the present invention, the sunscreen or daily care composition comprises
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives;
   wherein the composition does not comprise ethylhexyl-2-cyano-3,3-diphenyl-acrylate (INCI octocrylene).

In a second embodiment, the present invention refers to a sunscreen or daily care composition in the form of an emulsion, comprising
(i) hexyl 2-[4-(diethylamino)-2-hydroxybenzoyl]benzoate (INCI diethylamino hydroxybenzoyl hexyl benzoate) in an amount of from 2.5% to 9% by weight, based on the total weight of the composition; and
(ii) at least one organic particulate UV filter; and
(iii) at least one UV filter selected from triazine derivatives selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone) and combinations thereof;
   wherein the composition does not comprise ethylhexyl-2-cyano-3,3-diphenyl-acrylate (INCI octocrylene).

In another embodiment of the present invention, the sunscreen or daily care composition as defined above does not comprise 2-ethylhexyl-(2E)-3-(4-methoxyphenyl)acrylate (INCI ethylhexyl methoxy cinnamate).

In yet another embodiment of the present invention, the sunscreen or daily care composition as defined above is free of parabens. In this connection, it is to be understood that parabens are also known by the synonyms parahydroxybenzoate, oxybenzoates, oxybenzoic acid, hydroxybenzoic acid, and hydroxybenzoate, which are also excluded from the sunscreen or daily care composition according to the present invention.

In yet another embodiment of the present invention, the sunscreen or daily care composition as defined above is free of phenoxyethanol.

In yet another embodiment of the present invention, the sunscreen or daily care composition as defined above is free of (RS)-2-ethylhexyl-2-hydroxybenzoate (INCI ethylhexyl salicylate). In connection with the above embodiments, it is to be understood that free of means that the composition does not comprise the above-defined compounds or substances. In particular, it is to be understood that the composition does not comprise the above-defined compounds or substances in an amount of more than 0.8% by weight respectively, based on the total weight of the composition. Furthermore, it is to be understood that the composition does not comprise the above-defined compounds or substances in an amount of more than 0.5% by weight respectively.

In a preferred embodiment of the present invention, the sunscreen or daily care composition as defined above does not comprise each of the compounds or substances as defined above at all.

In connection with the above embodiments, it is to be understood that the sunscreen or daily care composition as defined above according to the present invention is free of the substances as defined above or the combinations as defined below.

Thus, in one preferred embodiment of the present invention, the sunscreen or daily care composition as defined above is free of
ethylhexyl-2-cyano-3,3-diphenyl-acrylate (INCI octocrylene), and
2-ethylhexyl-(2E)-3-(4-methoxyphenyl)acrylate (INCI ethylhexyl methoxy cinnamate).

In another preferred embodiment of the present invention, the sunscreen or daily care composition as defined above is free of
ethylhexyl-2-cyano-3,3-diphenyl-acrylate (INCI octocrylene), and
parabens.

In another preferred embodiment of the present invention, the sunscreen or daily care composition as defined above is free of
ethylhexyl-2-cyano-3,3-diphenyl-acrylate (INCI octocrylene), and
phenoxyethanol.

In another preferred embodiment of the present invention, the sunscreen or daily care composition as defined above is free of
ethylhexyl-2-cyano-3,3-diphenyl-acrylate (INCI octocrylene), and
ethylhexyl salicylate.

In another preferred embodiment of the present invention, the sunscreen or daily care composition as defined above is free of
ethylhexyl-2-cyano-3,3-diphenyl-acrylate (INCI octocrylene),
2-ethylhexyl-(2E)-3-(4-methoxyphenyl)acrylate (INCI ethylhexyl methoxy cinnamate), and
parabens.

In another preferred embodiment of the present invention, the sunscreen or daily care composition as defined above is free of
ethylhexyl-2-cyano-3,3-diphenyl-acrylate (INCI octocrylene),
2-ethylhexyl-(2E)-3-(4-methoxyphenyl)acrylate (INCI ethylhexyl methoxy cinnamate), and
phenoxyethanol.

In another preferred embodiment of the present invention, the sunscreen or daily care composition as defined above is free of
ethylhexyl-2-cyano-3,3-diphenyl-acrylate (INCI octocrylene),
2-ethylhexyl-(2E)-3-(4-methoxyphenyl)acrylate (INCI ethylhexyl methoxy cinnamate), and
ethylhexyl salicylate.

In another preferred embodiment of the present invention, the sunscreen or daily care composition as defined above is free of
parabens,
phenoxyethanol, and
ethylhexyl-2-cyano-3,3-diphenyl-acrylate (INCI octocrylene).

In another preferred embodiment of the present invention, the sunscreen or daily care composition as defined above is free of
parabens,
ethylhexyl salicylate, and
ethylhexyl-2-cyano-3,3-diphenyl-acrylate (INCI octocrylene).

In another preferred embodiment of the present invention, the sunscreen or daily care composition as defined above is free of
ethylhexyl salicylate,
phenoxyethanol, and
ethylhexyl-2-cyano-3,3-diphenyl-acrylate (INCI octocrylene).

In another preferred embodiment of the present invention, the sunscreen or daily care composition as defined above is free of
ethylhexyl-2-cyano-3,3-diphenyl-acrylate (INCI octocrylene),
2-ethylhexyl-(2E)-3-(4-methoxyphenyl)acrylate (INCI ethylhexyl methoxy cinnamate),
parabens, and
phenoxyethanol.

In another preferred embodiment of the present invention, the sunscreen or daily care composition as defined above is free of
ethylhexyl-2-cyano-3,3-diphenyl-acrylate (INCI octocrylene),
2-ethylhexyl-(2E)-3-(4-methoxyphenyl)acrylate (INCI ethylhexyl methoxy cinnamate),
parabens, and
ethylhexyl salicylate.

In another preferred embodiment of the present invention, the sunscreen or daily care composition as defined above is free of
ethylhexyl-2-cyano-3,3-diphenyl-acrylate (INCI octocrylene),
2-ethylhexyl-(2E)-3-(4-methoxyphenyl)acrylate (INCI ethylhexyl methoxy cinnamate),
phenoxyethanol, and
ethylhexyl salicylate.

In another preferred embodiment of the present invention, the sunscreen or daily care composition as defined above is free of
ethylhexyl-2-cyano-3,3-diphenyl-acrylate (INCI octocrylene),
parabens,
phenoxyethanol, and
ethylhexyl salicylate.

In a more preferred embodiment of the present invention, the sunscreen or daily care composition as defined above is free of
ethylhexyl-2-cyano-3,3-diphenyl-acrylate (INCI octocrylene),
2-ethylhexyl-(2E)-3-(4-methoxyphenyl)acrylate (INCI ethylhexyl methoxy cinnamate),
parabens,
phenoxyethanol, and
ethylhexyl salicylate.

In connection with the above preferred embodiments, it is to be understood that free of means that the composition does not comprise the substances or combinations thereof as defined above. In particular, it is to be understood that the composition does not comprise the above-defined substances or combinations thereof in an overall amount of more than 0.8% by weight, based on the total weight of the composition. Furthermore, it is to be understood that the composition does not comprise the above-defined substances or combinations thereof in an overall amount of more than 0.5% by weight.

In a preferred embodiment of the present invention, the sunscreen or daily care composition does not comprise the above-defined substances or combinations thereof at all.

In one embodiment of the present invention, the sunscreen or daily care composition as defined above further comprises at least one emollient with a polarity index of <30 mN/m.

In a preferred embodiment of the present invention, the sunscreen or daily care composition as defined above further comprises at least one emollient with a polarity index of <30 mN/m selected from
esters of linear or branched fatty acids with linear or branched fatty alcohols;
esters of aromatic carboxylic acids with linear or branched fatty alcohols;
di- and tricarboxylic acid esters with linear or branched alcohols;
esters of hydroxycarboxylic acids with linear or branched fatty alcohols;
esters of linear or branched fatty acids with polyhydric alcohol;
mono-, di-, tri-glycerides based on C₆-C₁₈ fatty acids;
guerbet alcohols.

In a more preferred embodiment of the present invention, the sunscreen or daily care composition as defined above further comprises at least one emollient with a polarity index of <30 mN/m selected from the group consisting of C₁₂-C₁₅ alkyl benzoate, caprylic/capric triglyceride, butylene glycol dicaprylate/dicaprate, propylene glycol dicaprylate/dicaprate, diisopropyl sebacate, octyldodecanol, isopropyl palmitate, isopropyl myristate, dicaprylyl carbonate, phenethyl benzoate, dibutyl adipate, diisopropyl adipate, triethyl citrate and tributyl citrate.

In a preferred embodiment, the sunscreen or daily care composition comprises the at least one emollient in an amount of from 1% to 20% by weight, preferably in an amount of from 2% to 15% by weight, based on the total weight of the sunscreen or daily care composition. It is to be understood that these amounts refer to each individual emollient in the sunscreen or daily care composition. Thus, each individual emollient in the sunscreen or daily care composition is preferably present in an amount of from 1% to 20% by weight, preferably in an amount of from 2% to 15% by weight, based on the total weight of the sunscreen or daily care composition. If two or more emollients are present in the sunscreen or daily care composition, the overall amount of emollients may preferably be in the range of from 1% to 35% by weight, preferably from 2% to 25% by weight, based on the total weight of the sunscreen or daily care composition.

In connection with the above preferred embodiments regarding the addition of the at least one emollient with a polarity index of <30 mN/m, it has surprisingly been found by the inventors of the present invention that the addition of at least one emollient as defined above enhances the solubility properties and performance of the UV filters as defined above in an octocrylene free sunscreen or daily care composition. In this connection, it is to be understood that the wavelength of maximum absorbance (λmax) is an indicator to describe the performance of a UV filter molecule. It gives the wavelength at which the absorbance is at maximum.

In another preferred embodiment of the present invention, the sunscreen or daily care composition has an UV-A protection, which is at least one third of the sun protection factor as defined above. For example, if the sun protection factor of a sunscreen composition is 30, the UV-A protection of the given sunscreen composition has to be at least 10. In particular, in connection with the UVA protection, the sunscreen or daily care composition comprises
(i) hexyl 2-[4-(diethylamino)-2-hydroxybenzoyl]benzoate (INCI diethylamino hydroxybenzoyl hexyl benzoate); and
(ii) 2,2′-methylene bis[6-(2H-benzotriazol-2-yl)-4-(1,1,3,3-tetramethylbutyl)phenol] (INCI methylene bis-benzotriazolyl tetramethylbutylphenol, MBBT), 2,4,6-tris(biphenyl-4-yl)-1,3,5-triazine (INCI trisbiphenyl triazine, TBPT); and
(iii) 4,4′,4″-(1,3,5-triazine-2,4,6-triyltriimino)tris-benzoic acid-tris(2-ethylhexyl)ester (INCI ethylhexyl triazone, EHT), 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine, BEMT);
   wherein the composition does not comprise ethylhexyl-2-cyano-3,3-diphenyl-acrylate (INCI octocrylene).

In connection with the above preferred embodiment regarding the UV-A protection, which is at least one third of the sun protection factor as defined above, it has surprisingly been found by the inventors of the present invention that the presence of the organic particulate UV filter 2,4,6-tris(biphenyl-4-yl)-1,3,5-triazine (INCI trisbiphenyl triazine, TBPT) in the sunscreen composition as defined above, enhances the sun protection factor while at the same time provides a UV-A protection which is at least one third of the sun protection factor (SPF). In other words, this means that the presence of the organic particulate UV filter trisbiphenyl triazine enhances the SPF without unbalancing the absorbance in the UV-A region and therefore providing a UV-A protection, which is at least one third of the sun protection factor.

Furthermore, in connection with the above preferred embodiment regarding the UVA protection, it is to be understood that the UVA protection by a sunscreen or daily care composition can be further determined by using the so-called Boots Star Rating System. The method requires that the transmission of ultraviolet (UV) radiation through a sunscreen product is measured throughout the terrestrial UV range before and after exposure to a fixed dose of simulated sunlight UV. For each sunscreen product test, it is necessary to calculate two separate UVA:UVB ratios. The measurement of the initial UVA:UVB absorbance ratio, requires a transmission measurement through the sunscreen sample at least fifteen minutes and no more than 30 minutes after application of the sunscreen sample to the substrate. The second UVA:UVB absorbance ratio measurement is the post absorbance measurement, which takes into account any photo-degradation of the UV filter of the sunscreen sample. Therefore, the sunscreen sample of the initial UVA:UVB ratio is exposed to a fixed dose of 17.5 J/cm² of UV radiation. The total irradiance (290 to 400 nm) is comprised between 45 and 90 W/m²; the UVA (320 to 400 nm) irradiance is comprised between 90 and 97% of the total irradiance. This dose approximately refers to the sunlight UV dose that might fall on an unshaded, horizontal surface when exposed to one hour of midday, midsummer sunshine. The photostability of the sunscreen product is further defined as the retention of at least 95% of the original UVA:UVB ratio. If less than 5% degradation are observed, the sunscreen product is sufficiently photostable and is labelled with the initial UVA:UVB ratio. In contrast, if the UVA:UVB ratio decreases by more than 5% than the product is labelled with the post-exposure UVA:UVB ratio. The star rating rates are further allocated on the basis of the initial and the post-exposure UVA:UVB ratio and ranges of from 3 to 5 stars.

Thus, in a particularly preferred embodiment, the present invention relates to a sunscreen composition comprising
(i) diethylamino hydroxybenzoyl hexyl benzoate; and
(ii) bis-ethylhexyloxyphenol methoxyphenyl triazine; and
(iii) ethylhexyl triazone; and
(iv) methylene bis-benzotriazolyl tetramethylbutylphenol and/or trisbiphenyl triazine,
   wherein the sunscreen composition has an in vitro Boots rating UVA:UVB ratio of at least 0.9 before and 0.86 after an UV irradiation of 17.5 J/cm².

In connection with the present invention, in particular in connection with the first and second embodiment, the following preferences regarding the UV filters are relevant in connection with the above listed embodiments of the invention.

In one embodiment of the present invention, the sunscreen or daily care composition as defined above comprises
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives;

wherein the composition does not comprise ethylhexyl-2-cyano-3,3-diphenyl-acrylate (INCI octocrylene).

In a preferred embodiment of the present invention, the sunscreen or daily care composition as defined above comprises the at least one organic particulate UV filter, wherein “at least one organic particulate UV filter” may preferably refer to from 1 to 3 organic particulate UV filter. In another preferred embodiment of the present invention, the sunscreen or daily care composition as defined above comprises the at least one organic particulate UV filter selected from the group consisting of 2,2′-methylene bis[6-(2H-benzotriazol-2-yl)-4-(1,1,3,3-tetramethylbutyl)phenol] (INCI methylene bis-benzotriazolyl tetramethylbutylphenol, MBBT), 2,4,6-tris(biphenyl-4-yl)-1,3,5-triazine (INCI trisbiphenyl triazine, TBPT), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine, BDBP), 5,6,5′,6′-tetraphenyl-3-3′-(1,4-phenylene)bis(1,2,4-triazine) (INCI phenylene bis-diphenyltriazine, PBT), micronized 1,4-di(benzoxazole-2′-yl)benzene and combinations thereof.

In a more preferred embodiment of the present invention, the sunscreen or daily care composition as defined above comprises the at least one organic particulate UV filter selected from the group consisting of 2,2′-methylene bis[6-(2H-benzotriazol-2-yl)-4-(1,1,3,3-tetramethylbutyl)phenol] (INCI methylene bis-benzotriazolyl tetramethylbutylphenol, MBBT), 2,4,6-tris(biphenyl-4-yl)-1,3,5-triazine (INCI trisbiphenyl triazine, TBPT), 5,6,5′,6′-tetraphenyl-3-3′-(1,4-phenylene)bis(1,2,4-triazine) (INCI phenylene bis-diphenyltriazine, PBT) and combinations thereof. In connection with the above preferred embodiments, it is to be understood that, if two or more organic particulate UV filters are present, also combinations of organic particulate UV filters are possible, e.g., the combination of methylene bis-benzotriazolyl tetramethylbutylphenol and trisbiphenyl triazine.

If combinations of 2 organic particulate UV filters are present, the following combinations are individually preferred:
methylene bis-benzotriazolyl tetramethylbutylphenol (MBBT) and trisbiphenyl triazine (TBPT);
methylene bis-benzotriazolyl tetramethylbutylphenol (MBBT) and phenylene bis-diphenyltriazine (PBT);
methylene bis-benzotriazolyl tetramethylbutylphenol (MBBT) and bis-(diethylaminohydroxybenzoyl benzoyl) piperazine (BDBP);
trisbiphenyl triazine (TBPT) and phenylene bis-diphenyltriazine (PBT);
trisbiphenyl triazine (TBPT) and bis-(diethylaminohydroxybenzoyl benzoyl) piperazine (BDBP);
phenylene bis-diphenyltriazine (PBT) and bis-(diethylaminohydroxybenzoyl benzoyl) piperazine (BDBP).

If combinations of 3 organic particulate UV filters are present, the following combinations are individually preferred:
methylene bis-benzotriazolyl tetramethylbutylphenol (MBBT), trisbiphenyl triazine (TBPT) and bis-(diethylaminohydroxybenzoyl benzoyl) piperazine (BDBP);
methylene bis-benzotriazolyl tetramethylbutylphenol (MBBT), trisbiphenyl triazine (TBPT) and phenylene bis-diphenyltriazine (PBT);
methylene bis-benzotriazolyl tetramethylbutylphenol (MBBT), bis-(diethylaminohydroxybenzoyl benzoyl) piperazine (BDBP) and phenylene bis-diphenyltriazine (PBT);
trisbiphenyl triazine (TBPT), phenylene bis-diphenyltriazine (PBT) and bis-(diethylaminohydroxybenzoyl benzoyl) piperazine (BDBP).

In a preferred embodiment, the sunscreen or daily care composition comprises the at least one organic particulate UV filter in an amount of from 0.5% to 10% by weight, preferably in an amount of from 1% to 5% by weight, based on the total weight of the sunscreen or daily care composition. It is to be understood that these amounts refer to each individual organic particulate UV filter in the sunscreen or daily care composition. Thus, each individual organic particulate UV filter in the sunscreen or daily care composition is preferably present in an amount of from 0.5% to 10% by weight, preferably in an amount of from 1% to 5% by weight, based on the total weight of the sunscreen or daily care composition. If two or more organic particulate UV filters are present in the sunscreen or daily care composition, the overall amount of organic particulate UV filters may preferably be in the range of from 1% to 30% by weight, preferably from 3% to 30% by weight, based on the total weight of the sunscreen or daily care composition.

It is to be understood that the above preferred embodiments refer to the at least one organic particulate UV filter, which is present in the sunscreen or daily care composition in addition to hexyl 2-[4-(diethylamino)-2-hydroxybenzoyl]benzoate (INCI diethylamino hydroxybenzoyl hexyl benzoate) and the at least one UV filter selected from triazine derivatives.

In a preferred embodiment of the present invention, the sunscreen or daily care composition as defined above comprises the at least one UV filter selected from triazine derivatives, wherein “at least one UV filter selected from triazine derivatives” may preferably refer to from 1 to 3 UV filter selected from triazine derivatives.

In a more preferred embodiment of the present invention, the sunscreen or daily care composition comprises the at least one UV filter selected from triazine derivatives selected from the group consisting of 4,4′,4″-(1,3,5-triazine-2,4,6-triyltriimino)tris-benzoic acid-tris(2-ethylhexyl)ester (INCI ethylhexyl triazone, EHT), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone, DBT), 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine, BEMT) and combinations thereof. In connection with the above preferred embodiments, it is to be understood that, if two or more UV filters selected from triazine derivatives are present, also combinations of UV filters selected from triazine derivatives are possible, e.g., the combination of ethylhexyl triazone and bis-ethylhexyloxyphenol methoxyphenyl triazine.

If combinations of 2 UV filters selected from triazine derivatives are present, the following combinations are individually preferred:
ethylhexyl triazone (EHT) and diethylhexyl-butamidotriazone (DBT);
ethylhexyl triazone (EHT) and bis-ethylhexyloxyphenol methoxyphenyl triazine (BEMT); and
diethylhexyl-butamidotriazone (DBT) and bis-ethylhexyloxyphenol methoxyphenyl triazine (BEMT).

If combinations of 3 UV filters selected from triazine derivatives are present, the combination of ethylhexyl triazone (EHT), diethylhexyl-butamidotriazone (DBT) and bis-ethylhexyloxyphenol methoxyphenyl triazine (BEMT) is preferred.

In a particularly preferred embodiment of the present invention, the sunscreen or daily care composition comprises the at least one UV filter selected from triazine derivatives selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone) and combinations thereof. In another preferred embodiment of the present invention, the sunscreen or daily care composition comprises the at least one UV filter selected from triazine derivatives in an amount of from 0.5% to 5% by weight, preferably in an amount of from 0.8% to 3% by weight, based on the total weight of the sunscreen or daily care composition. It is to be understood that these amounts refer to each individual UV filter selected from triazine derivatives in the sunscreen or daily care composition. Thus, each individual UV filter selected from triazine derivatives in the sunscreen or daily care composition is preferably present in an amount of from 0.5% to 5% by weight, preferably in an amount of from 0.8% to 3% by weight, based on the total weight of the sunscreen or daily care composition. If two or more UV filter selected from triazine derivatives are present in the sunscreen or daily care composition, the overall amount of UV filter selected from triazine derivatives may preferably be in the range of from 1% to 10% by weight, preferably from 2.5% to 10% by weight, based on the total weight of the sunscreen or daily care composition.

It is to be understood that the above preferred embodiments refer to the at least one UV filter selected from triazine derivatives, which is present in the sunscreen or daily care composition in addition to hexyl 2-[4-(diethylamino)-2-hydroxybenzoyl]benzoate (INCI diethylamino hydroxybenzoyl hexyl benzoate) and the at least one organic particulate UV filter.

In connection with the above preferred embodiments regarding the UV filters, it is to be understood that also combinations of one or more organic particulate UV filter with combinations of one or more UV filter selected from triazine derivatives in addition to hexyl 2-[4-(diethylamino)-2-hydroxybenzoyl]benzoate (INCI diethylamino hydroxybenzoyl hexyl benzoate) are part of the invention.

In another preferred embodiment of the present invention, the sunscreen or daily care composition as defined above comprises hexyl 2-[4-(diethylamino)-2-hydroxybenzoyl]benzoate (INCI diethylamino hydroxybenzoyl hexyl benzoate) in an amount of from 0.5% to 10% by weight, based on the total weight of the composition.

In another preferred embodiment of the present invention, the sunscreen or daily care composition as defined above comprises hexyl 2-[4-(diethylamino)-2-hydroxybenzoyl]benzoate (INCI diethylamino hydroxybenzoyl hexyl benzoate) in an amount of from 2.5% to 9% by weight, based on the total weight of the composition.

In another embodiment of the present invention, the sunscreen or daily care composition as defined above comprises hexyl 2-[4-(diethylamino)-2-hydroxybenzoyl]benzoate (INCI diethylamino hydroxybenzoyl hexyl benzoate) in an amount of from 3% to 8% by weight, based on the total weight of the composition.

In another embodiment of the present invention, the sunscreen or daily care composition comprises hexyl 2-[4-(diethylamino)-2-hydroxybenzoyl]benzoate (INCI diethylamino hydroxybenzoyl hexyl benzoate) in an amount of from 4% to 7% by weight, based on the total weight of the composition.

In connection with the above preferred embodiments, it is to be understood that the total weight of the sunscreen or daily care composition refers to the sunscreen or daily care composition as defined above, wherein the sunscreen composition may comprise at least one additive.

In one embodiment, the at least one additive is selected from the group consisting of emulsifier, viscosity regulators (thickeners), sensory enhancers, adjuvants, preservatives, and combinations thereof.

Preferred emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives such as polysorbate-n, PEG-10 sorbitan laurate;
fatty alcohol polyglycolethers and fatty acid polyglycolethers such as ceteareth-20, beheneth-25, steareth-2, PEG-100 stearate;
glycerides of fatty acids such as glyceryl stearate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate;
fatty acid esters of polyglyceryl such as polyglyceryl-3-diisostearate, polyglyceryl-2-dipolyhydroxystearate;
oxyalkenylated organomodified silicone/polysiloxane/polyalkyl/polyether copolymers and derivatives.

Preferred thickeners include
fatty alcohols such as cetyl alcohol, cetearyl alcohol, stearyl alcohol;
fatty acids such as stearic acid;
fatty acid esters such as myristyl stearate;
waxes such as beeswax, carnauba wax, microcrystalline wax, ceresin, ozocerite;
polysaccharides or derivatives such as xanthan gum, guar gum, agar gum, alginates, gellan gum, carraghenan;
polyacrylates or homopolymers of reticulated acrylic acids or polyacrylamides such as carbomers, acrylate copolymers, acrylate/C₁₀-C₃₀-alkyl acrylate crosspolymer, acrylate/beheneth-25 methacrylate copolymer;
silicate derivatives such as magnesium silicates;
cellulose derivatives such as hydroxypropyl cellulose.

Preferred sensory enhancers include
polyamide derivatives such as nylon-12;
polymethyl methacrylates;
silica;
mica;
polymethylsilsesquioxane;
polyethylene;
starch derivatives such as aluminum starch octenylsuccinate;
dimethicone derivatives;
boron nitride;
HDI/trimethylol hexyllactone crosspolymer.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
caprylyl glycol;
hydroxyacetophenone;
caprylhydroxymic acid;
propellants such as propane, butane, isobutene, dimethyl ether;
styrene/PVP or styrene acrylamide copolymers;
insect repellants such as butylacetylaminopropionate.

Preferred preservatives include
benzyl alcohol;
zingerone.

In another embodiment of the present invention, the sunscreen or daily care composition comprises at least one perfume.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmethylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylsalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

In connection with the at least one perfume present in the sunscreen or daily care composition, it has surprisingly been found that in case the composition comprises a UV filter in combination with a perfume, oxidative degeneration is reduced. In particular, it has surprisingly been found that the UV filter DHHB in the presence of at least one perfume reduces the oxidative degeneration.

In connection with the above preferred embodiments, it is to be understood that if the sunscreen or daily care composition comprises two or more additives, combinations of the additives as defined above are also part of the invention.

In connection with the above preferred and particularly preferred embodiments, it is to be understood that the sunscreen or daily care composition, in its final formulation, may exist in a wide variety of presentation forms, which include
liquid preparations as a W/O, O/W, O/W/O, W/O/W or PIT emulsion and all kinds of micro emulsions;
gels;
oil, cream, milk or lotion;
powder, a lacquer, a tablet or make-up;
sticks;
sprays (spray with propellant gas or pump-action spray) or an aerosol;
foams;
pastes.

In connection with the above preferred and particularly preferred embodiments, it is to be understood that the sunscreen or daily care composition, in its final formulation, preferably exists in the form of an emulsion. Preferred emulsions according to the present application include W/O, O/W, O/W/O, W/O/W or PIT emulsions.

In connection with the present invention, the following preferences regarding the use of the sunscreen composition according to the embodiments as defined above to reduce fabric staining and/or to facilitate the washability of the sunscreen or daily care composition from textiles are relevant.

As indicated above, the present invention relates in one embodiment to the use of a sunscreen or daily care composition, in the form of an emulsion, as defined above, in particular as defined with regard to the first and second embodiment, to reduce fabric staining of a sunscreen or daily care composition.

Fabric staining caused by sunscreen compositions is preferably measured by the so-called L*a*b method. With this method, the color difference between two samples can be identified based on numerical values. The advantage of this method is that it is not dependent on the device used for measuring the colors. Instead, the color difference can be determined based on numerical values for different colors. As used herein, the color difference between a sunscreen composition stain on a textile and the textile alone is determined in order to determine fabric staining of a sunscreen composition. Different sunscreen compositions and their fabric staining properties can thus be measured by comparing the color of their stains on a textile with the color of the textile alone. The lower the color difference is, the less fabric staining is observed. Similarly, the color difference between a sunscreen composition stain on a textile after one or more washing cycles and the textile alone can be determined in order to determine a potential reduction of fabric staining after washing, i.e. the washability. The color difference is indicated in absolute color coordinates and can be referred to as Delta A. First, the colors of the two samples are measured. Then, the difference can be calculated using the resulting colorimetric values, the CIE L*a*b coordinates. The L*a*b color space, defined by the Commission Internationale de l'Eclairage (CIE), was modeled after a color-opponent theory. This theory states that two colors cannot be red and green at the same time or yellow and blue at the same time. The following formula is used to determine the total color difference between two samples:

ΔE*=[ΔL*2+Δa*2+Δb*2]1/2

In this regard, L* indicates lightness, a* is the red/green coordinate, and b* is the yellow/blue coordinate. Delta values for L* (ΔL*), a* (Δa*), and b* (Δb*) can be positive or negative. The total difference, given as ΔE* is always positive.

ΔL*(L*sample 1−L*sample 2)=difference in lightness and darkness (+ is lighter, − is darker).

Δa*(a*sample 1−a*sample 2)=difference in red and green (+ is redder, − is greener)

Δb*(b*sample 1−b*sample 2)=difference in yellow and green (+ is yellower, − is greener)

ΔE*=total color difference.

In order to determine fabric staining according to the present invention, the color of the stain on a textile is compared to the color of the textile alone. Preferably, a white textile is used. In case the stain is only yellow, ΔE*≃Δb*, wherein the visibility of the sunscreen composition stain is expressed by Δb*. As the color of a sunscreen composition stain is typically yellow, ΔE* is considered to be identical to Δb* for the purpose of the present invention.

In another embodiment, the present invention relates to the use of a sunscreen or daily care composition, in the form of an emulsion, as defined above, in particular as defined with regard to the first and second embodiment, to facilitate the washability of a sunscreen or daily care composition from textiles.

As used herein, the washability refers to the extent to which sunscreen composition stains on textiles can be removed by washing. The washability, i.e. the removability, of a sunscreen composition stain can be determined by determining the color difference of the stain according to the L*a*b* method described above before and after one or more washing cycles.

In connection with the above embodiments, it is to be understood that the following sunscreen or daily care composition is preferred with regard to a reduction of fabric staining and to facilitate the washability of the sunscreen or daily care composition from textiles.

In one embodiment of the uses of the present invention, the sunscreen or daily care composition comprises
(i) hexyl 2-[4-(diethylamino)-2-hydroxybenzoyl]benzoate (INCI diethylamino hydroxybenzoyl hexyl benzoate); and
(ii) at least one organic particulate UV filter, wherein the at least one organic particulate UV filter is preferably 2,2′-methylene bis[6-(2H-benzotriazol-2-yl)-4-(1,1,3,3-tetramethylbutyl)phenol] (INCI methylene bis-benzotriazolyl tetramethylbutylphenol); and
(iii) at least one UV filter selected from triazine derivatives;
   wherein the composition does not comprise ethylhexyl-2-cyano-3,3-diphenyl-acrylate (INCI octocrylene).

In this connection, it is to be understood that the at least one UV filter selected from triazine derivatives is as defined above. Furthermore, it is to be understood that the composition as defined above preferably does not comprise 2-ethylhexyl-(2E)-3-(4-methoxyphenyl)acrylate (INCI ethylhexyl methoxy cinnamate) and is free of parabens.

The present invention is further illustrated by the following examples.

### EXAMPLES

Process of Manufacture of Sunscreen Compositions

The ingredients of part A, as well as the ingredients of part B as provided below in Tables 1.1 and 1.2 for each tested sunscreen composition were combined and heated under stirring to 75-80° C. respectively, wherein part A was added to part B under stirring and was further homogenized. Subsequently, the sunscreen composition was cooled down to room temperature under stirring and part C was added if present.

All amounts referred to in the following tables refer to the respective amounts in % by weight, based on the total weight of the composition.

TABLE 1.1

| | | | |
|---|---|---|---|
| | Base | Comparative | Comparative |
| Ingredient (Trade Name) | composition | composition 1a | composition 1b |

Part A

| | | | |
|---|---|---|---|
| C₁₂-C₁₅ alkyl benzoate (Cetiol AB) | 3.00 | 3.00 | 3.00 |
| Dibutyl adipate (Cetiol B) | 17.00 | 17.00 | 17.00 |
| Cetearyl alcohol (Lanette O) | 2.50 | 2.50 | 2.50 |
| EHT (Uvinul T 150) | 3.00 | 3.00 | 3.00 |
| DHHB (Uvinul A Plus) | 4.00 | 4.00 | 4.00 |
| BEMT (Tinosorb S) | 1.00 | 1.00 | 1.00 |
| Titanium Dioxide | — | 3.00 | — |
| PBSA | — | — | 3.00 |
| EHMC (Uvinul MC 80) | — | — | — |
| DBT (Uvasorb HEB) | — | — | — |

Part B

| | | | |
|---|---|---|---|
| Water | qsp 100% | qsp 100% | qsp 100% |
| Glycerin | 3.00 | 3.00 | 3.00 |
| Disodium EDTA (EDTA BD) | 0.20 | 0.20 | 0.20 |
| Xanthan Gum (Rheocare XG) | 0.15 | 0.15 | 0.15 |
| Potassium cetyl phosphate | 2.50 | 2.50 | 2.50 |
| (Amphisol K) | | | |

Part C

| | | | |
|---|---|---|---|
| TBPT (Tinosorb A2B) | — | — | — |

TABLE 1.2

| | | | |
|---|---|---|---|
| | Comparative | Comparative | |
| Ingredient (Trade Name) | composition 1c | composition 1d | Composition 1 |

Part A

| | | | |
|---|---|---|---|
| C₁₂-C₁₅ alkyl benzoate | 3.00 | 3.00 | 3.00 |
| (Cetiol AB) | | | |
| Dibutyl adipate (Cetiol B) | 17.00 | 17.00 | 17.00 |
| Cetearyl alcohol (Lanette O) | 2.50 | 2.50 | 2.50 |
| EHT (Uvinul T 150) | 3.00 | 3.00 | 3.00 |
| DHHB (Uvinul A Plus) | 4.00 | 4.00 | 4.00 |
| BEMT (Tinosorb S) | 1.00 | 1.00 | 1.00 |
| Titanium Dioxide | — | — | — |
| PBSA | | | |
| EHMC (Uvinul MC 80) | 3.00 | — | — |
| DBT (Uvasorb HEB) | — | 3.00 | — |

Part B

| | | | |
|---|---|---|---|
| Water | qsp 100% | qsp 100% | qsp 100% |
| Glycerin | 3.00 | 3.00 | 3.00 |
| Disodium EDTA (EDTA BD) | 0.20 | 0.20 | 0.20 |
| Xanthan Gum (Rheocare XG) | 0.15 | 0.15 | 0.15 |
| Potassium cetyl phosphate | 2.50 | 2.50 | 2.50 |
| (Amphisol K) | | | |

Part C

| | | | |
|---|---|---|---|
| TBPT (Tinosorb A2B) | — | — | 3.00* |

*Active amount

SPF and UV-A Measurements:

The SPF and UVA protection of the base composition, comparative compositions and composition 1 were determined according to standard procedures (ISO 24444, ISO 24443).

TABLE 2

| | SPF in vivo | UVA-PF/SPF |
|---|---|---|
| Base composition | 17.2 | >1/3 |
| Comparative composition 1a | 25.1 | <1/3 |
| Comparative composition 1b | 28.8 | <1/3 |
| Comparative composition 1c | 28.2 | <1/3 |
| Comparative composition 1d | 37.2 | <1/3 |
| Composition 1 | 43.0 | >1/3 |

Measurement of UVA Protection According to Boots Star Rating System

Process of Manufacture of Sunscreen Composition

The ingredients of part A, as well as the ingredients of part B as provided below in Table 3 were combined and heated under stirring to 75-80° C. respectively, wherein part A was added to part B under stirring and was further homogenized. Subsequently, the sunscreen composition was cooled down to room temperature and part C was added.

TABLE 3

| | |
|---|---|
| Ingredient (Trade Name) | Composition 2 |

Part A

| | |
|---|---|
| Succrose polystearate | 3.00 |
| (Emulgade Sucro) | |
| Disodium cetearyl sulfosuccinate | 1.00 |
| (Emulgin Prisma) | |
| Dibutyl adipate (Cetiol B) | 8.00 |
| C₁₂₋₁₅ alkyl benzoate (Cetiol AB) | 6.00 |
| Diisopropyl sebacate (Dib Dis) | 5.00 |
| Isopropyl myristate (IPM) | 3.00 |
| Cetearyl alcohol (Lanette O) | 2.00 |
| Ethylhexylglycerin (Sensiva SC 50) | 0.50 |
| DHHB (Uvinul A Plus) | 10.00 |
| BEMT (Tinosorb S) | 2.00 |
| EHT (Uvinul T150) | 2.50 |

Part B

| | |
|---|---|
| Water | qsp 100% |
| Glycerin | 6.20 |
| Xanthan Gum (Rheocare XGN) | 0.30 |

Part C

| | |
|---|---|
| MBBT (Tinosorb M)* | 3.00 |
| TBPT (Tinosorb A2B)* | 2.00 |

*The amount refers to active matter, 3% MBBT correspond to 6% of the commercial product Tinosorb M; 2% TBPT correspond to 4% of the commercial product Tinosorb A26.

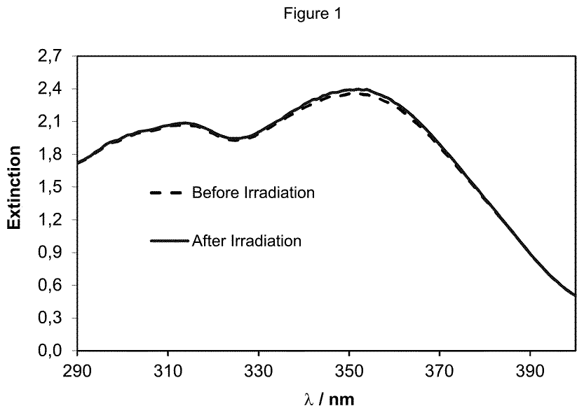

For each sunscreen composition, the UVA:UVB ratio is measured before and after irradiation with a fixed dose of 17.5 J/cm² of UV radiation. The measurement of the initial UVA:UVB absorbance ratio, requires a transmission measurement through the sunscreen sample at least fifteen minutes and no more than 30 minutes after application of the sunscreen sample to the substrate. The second UVA:UVB absorbance ratio measurement is the post absorbance measurement, which takes into account any photo-degradation of the UV filter of the sunscreen sample. Therefore, the sunscreen sample of the initial UVA:UVB ratio is exposed to a fixed dose of 17.5 J/cm² of UV radiation. The UVA:UVB ratio before and after irradiation is displayed below and in FIG. 1.

UVA: UVB ratio of composition 2 before irradiation: 0.90

UVA: UVB ratio of composition 2 after irradiation: 0.91

Fabric Staining Measurements

Process of Manufacture of Sunscreen Compositions

The ingredients of part A as well as the ingredients of part B as provided below in Table 4 for each tested sunscreen composition were combined and heated under stirring at 80° C. respectively wherein part A was added to part B under stirring and further homogenized. Part C was added and stirred and the sunscreen composition was cooled down to room temperature. Then, part D was added and stirred in.

TABLE 4

| | | |
|---|---|---|
| | Comparative | |
| Ingredient (Trade Name) | Composition 1e | Composition 3 |

Part A

| | | |
|---|---|---|
| Glyceryl stearate | 1.00 | 1.00 |
| Sodium Stearyl Glutamate | 0.30 | 0.30 |
| Stearyl Alcohol | 1.00 | 1.00 |
| Butylene Glycol Dicaprylate/Dicaprate | 4.00 | 4.00 |
| Dibutyl adipate (Cetiol B) | 3.00 | 3.00 |
| C12-15 Alkyl Benzoate (Cetiol AB) | 5.00 | 5.00 |
| Phenoxyethanol | 1.00 | 1.00 |
| VP/Hexadecene Copolymer | 0.50 | 0.50 |
| Silica Dimethyl Silylate | 1.00 | 1.00 |
| Butyl Methoxydibenzoylmethane | 4.75 | 1.00 |
| Ethylhexyl Salicylate | 4.75 | 4.75 |
| Ethylhexyl Triazone (Uvinul T 150) | 3.00 | 3.00 |
| Bis-Ethylhexyloxyphenol | 4.00 | — |
| Methoxyphenyl Triazine (Tinosorb S) | | |
| Diethylamino Hydroxybenzoyl Hexyl | 1.00 | 3.00 |
| Benzoate (Uvinul A Plus) | | |

Part B

| | | |
|---|---|---|
| Aqua | qsp 100% | qsp 100% |
| Glycerin | 7.50 | 7.50 |
| Acrylates/C10-30 Alkyl Acrylate | 0.10 | 0.10 |
| Crosspolymer | | |
| Xanthan gum | 0.40 | 0.40 |
| Dissodium EDTA | 0.20 | 0.20 |

Part C

| | | |
|---|---|---|
| Phenylbenzimidazole Sulfonic Acid | 1.00 | 1.00 |
| Water | 5.00 | 5.00 |
| Sodium Hydroxyde | qs | qs |

Part D

| | | |
|---|---|---|
| Methylene Bis-Benzotriazolyl | — | 4.00 |
| Tetramethylbutyl-phenol (nano)* | | |
| Ethanol | 3.00 | 3.00 |
| SPF kal. | 36 | 36 |
| UVA-PF kal. | 13.2 | 14.9 |

*50% water dispersion: 4% Methylene Bis-Benzotriazolyl Tetramethylbutylphenol corresponds to 8% of supplied product (Tinosorb M)

Fabric staining of Composition 3 as well as Comparative Composition 1e has been measured as described above. The results are shown in Table 5.

TABLE 5

| | Comparative | |
|---|---|---|
| | Composition 1e | Composition 3 |
| Δb* (before washing) | 10.9 | 10.3 |
| SD (before washing) | 0.04 | 0.17 |
| Δb* (after 1 cycle) | 7.30 | 6.00 |
| SD (after 1 cycle) | 0.04 | 0.04 |
| Δb* (after 3 cycles) | 3.90 | 2.80 |
| SD (after 3 cycles) | 0.09 | 0.12 |

As can be seen from the results above, comparative Composition 1e exhibits a stronger fabric staining than Composition 3. Inventive Composition 3, however, containing Methylene Bis-Benzotriazolyl Tetramethylbutylphenol (nano) instead of Bis-Ethylhexyloxyphenol Methoxyphenyl Triazine shows a significant fabric staining reduction.

Measurement of Interfacial Tension (Polarity Index)

Interfacial tension of the respective emollients as listed below was measured using a Tensimat Densimat TD2000.

TABLE 6.1

| | | polarity |
|---|---|---|
| | | index |
| | | [mN/m] |
| polarity index | hydrogenated | 45 |
| >30 mN/m | polyisobutene | |
| (reference | mineral oil | 43 |
| emollients) | cyclopentasiloxane | 32 |
| polarity index | octyldodecanol | 25 |
| <30 mN/m | butylene glycol | 22 |
| | dicaprylate/dicaprate | |
| | C12-15 alkyl benzoate | 22 |
| | caprylic capric triglyceride | 21 |
| | propylene glycol | 20 |
| | dicaprylate/dicaprate | |
| | diisopropyl sebacate | 19 |
| | phenethyl benzoate | 19 |
| | dibutyl adipate | 14 |
| | diisopropyl adipate | 12 |
| | triethyl citrate | 7 |
| | tributyl citrate | 10 |

UV Filter Performance

The wavelength of maximum absorbance (λmax) is an indicator to describe the performance of a UV filter molecule. It gives the wavelength at which the absorbance is at maximum. For a UVA filter, the wavelength of maximum absorbance should be close to 359 nm that corresponds to the apex of the PPD (persistent pigment darkening) effectiveness spectrum. The PPD effectiveness spectrum corresponds to the multiplication of the PPD (persistent pigment darkening) action spectrum with the light intensity (see ISO 24443).

The UV transmission spectrum of each DHHB—emollient mixture was measured from 290 to 400 nm using a UVNis spectrophotometer Perkin Elmer Lambda 25. Three stock solutions with a concentration of 1 mM of DHHB were prepared for each mixture. Further, three dilutions of each stock solution were prepared resulting in nine measurements in total per DHHB—emollient solution. Then, all solutions were filled in UV transparent quartz cuvette of 1 cm optical path-length for UV Transmission measurements. λmax values of DHHB

TABLE 6.2

| | | λmax |
|---|---|---|
| | | values [nm] |
| | | (DHHB) |
| polarity index | hydrogenated | 345 |
| >30 mN/m | polyisobutene | |
| (reference | cyclomethicone | 345 |
| emollients) | mineral oil | 346 |
| polarity index | phenethyl benzoate | 355 |
| <30 mN/m | dibutyl adipate | 351 |
| | C12-15 alkyl | 351 |
| | benzoate | |
| | diisopropyl adipate | 351 |
| | butylene glycol | 350 |
| | dicaprylate/dicaprate | |
| | caprylic capric | 350 |
| | triglyceride | |
| | diisopropyl sebacate | 350 |
| | octyldodecanol | 349 |
| | triethyl citrate | 353 |
| | tributyl citrate | 352 |

Solubility Measurements

0.02 g of UV Filter are added to 2 ml of the respective emollient as displayed below, previously filled into a 20 ml vial with cap. The vial is placed in a thermostated water bath (25° C.) and the blend is stirred for seven days. If the tested UV-filter is fully soluble, additional filter is added until precipitation is observed. After seven days, the sample is centrifuged for 30 minutes at 13000 rpm. If the supernatant is turbid, it is filtered through a 0.2 μm non steril Membrex 25 PET filter. Prior to the measurement, the samples were either diluted with a suitable solvent, or, in case of lower concentrations measured without further dilution in quartz cuvettes of 1 cm optical path-length. The concentration of UV-filter is then determined with UV/Vis-spectroscopy using a Perkin Elmer Lambda 20 device (according to Method A in Herzog B., Giesinger J., Schnyder M., SOFW Journal, 2013, 139 (7), pages 7-14.).

TABLE 6.3

| | | solubility [%] |
|---|---|---|
| | | (DHHB) |
| polarity index | mineral oil | <1 |
| >30 mN/m | hydrogenated | <1 |
| (reference | polyisobutene | |
| emollients) | cyclomethicone | <1 |
| polarity index | phenethyl benzoate | 57 |
| <30 mN/m | dibutyl adipate | 31 |
| | C12-15 alkyl | 22 |
| | benzoate | |
| | diisopropyl adipate | 21 |
| | butylene glycol | 23 |
| | dicaprylate/dicaprate | |
| | propylene glycol | 22 |
| | dicaprylate/dicaprate | |
| | caprylic capric | 14 |
| | triglyceride | |
| | diisopropyl sebacate | 41 |
| | octyldodecanol | 2.5 |
| | triethyl citrate | 29.0 |
| | tributyl citrate | 29.4 |

In a particularly preferred embodiment, the present invention refers to the following further items:
1. A sunscreen or daily care composition comprising
   (i) hexyl 2-[4-(diethylamino)-2-hydroxybenzoyl]benzoate (INCI diethylamino hydroxybenzoyl hexyl benzoate); and
   (ii) at least one organic particulate UV filter; and
   (iii) at least one UV filter selected from triazine derivatives;
      wherein the composition does not comprise ethylhexyl-2-cyano-3,3-diphenyl-acrylate (INCI octocrylene).
2. The sunscreen or daily care composition according to item 1, wherein the composition does not comprise 2-ethylhexyl-(2E)-3-(4-methoxyphenyl)acrylate (INCI ethylhexyl methoxy cinnamate).
3. The sunscreen or daily care composition according to item 1 or 2, wherein the composition is free of parabens.
4. The sunscreen or daily care composition according to any one of items 1 to 3, wherein the at least one UV filter selected from triazine derivatives is selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone), 2,4-bis-{[4-(2-ethyl-hexyloxy)-2-hydroxy]-phenyl}-6-(4-methoxyphenyl)-1,3,5 triazine (INCI bis-ethylhexyloxyphenol methoxyphenyl triazine) and combinations thereof.
5. The sunscreen or daily care composition according to any one of items 1 to 4, wherein the at least one organic particulate UV filter is selected from the group consisting of 2,2′-methylene bis[6-(2H-benzotriazol-2-yl)-4-(1,1,3,3-tetramethylbutyl)phenol] (INCI methylene bis-benzotriazolyl tetramethylbutylphenol), 2,4,6-tris(biphenyl-4-yl)-1,3,5-triazine (INCI trisbiphenyl triazine), 1,1 ‘-(1,4-piperazinediyl)bis[1-[2-[4-(diethylamino)-2-hydroxybenzoyl]phenyl]-methanone (INCI bis-(diethylaminohydroxybenzoyl benzoyl) piperazine), 5,6,5’,6′-tetraphenyl-3-3′-(1,4-phenylene)bis(1,2,4-triazine) (INCI phenylene bis-diphenyltriazine) and combinations thereof.
6. The sunscreen or daily care composition according to any one of items 1 to 5, wherein the composition further comprises at least one emollient with a polarity index of <30 mN/m.
7. The sunscreen or daily care composition according to item 6, wherein the at least one emollient is selected from the group consisting of C₁₂-C₁₅ alkyl benzoate, caprylic/capric triglyceride, butylene glycol dicaprylate/dicaprate, propylene glycol dicaprylate/dicaprate, diisopropyl sebacate, octyldodecanol, isopropyl palmitate, isopropyl myristate, dicaprylyl carbonate, phenethyl benzoate, dibutyl adipate and diisopropyl adipate.
8. The sunscreen or daily care composition according to any one of items 1 to 7, wherein the composition comprises hexyl 2-[4-(diethylamino)-2-hydroxybenzoyl]benzoate (INCI diethylamino hydroxybenzoyl hexyl benzoate) in an amount of from 0.5% to 10% by weight, based on the total weight of the composition.
9. The sunscreen or daily care composition according to any one of items 1 to 8, wherein the composition comprises the at least one organic particulate UV filter in an amount of from 0.5% to 10% by weight, preferably in an amount of from 1% to 5% by weight, based on the total weight of the composition.
10. The sunscreen or daily care composition according to any one of items 1 to 9, wherein the composition comprises the at least one UV filter selected from triazine derivatives in an amount of from 0.5% to 5% by weight, preferably in an amount of from 0.8% to 3% by weight, based on the total weight of the composition.
11. The sunscreen or daily care composition according to any one of items 1 to 10, wherein the composition comprises perfume.
12. The sunscreen or daily care composition according to any one of items 1 to 11, wherein the composition is free of phenoxyethanol.
13. The sunscreen or daily care composition according to any one of items 1 to 12, wherein the composition has an UV-A protection, which is at least one third of the sun protection factor of the sunscreen composition.
14. Use of a composition as defined in any one of items 1 to 13 to reduce fabric staining of a sunscreen or daily care composition.
15. Use of a composition as defined in any one of items 1 to 13 to facilitate the washability of a sunscreen or daily care composition.

1.-15. (canceled)

16. A sunscreen or daily care composition, in the form of an emulsion, comprising
(i) hexyl 2-[4-(diethylamino)-2-hydroxybenzoyl]benzoate (INCI diethylamino hydroxybenzoyl hexyl benzoate) in an amount of from 2.5% to 9% by weight based on the total weight of the composition; and
(ii) at least one organic particulate UV filter; and
(iii) at least one UV filter selected from triazine derivatives selected from the group consisting of 4,4′,4″-(1,3,5-triazine-2,4,6-triyltriimino)tris-benzoic acid-tris(2-ethylhexyl)ester (INCI ethylhexyl triazone), 4,4′-[[6-[[4-[[(1,1-dimethylethyl)amino]carbonyl]phenyl]amino]-1,3,5-triazin-2,4-diyl]diimino]bis-benzoic acid-bis(2-ethylhexyl)ester (INCI diethylhexyl-butamidotriazone) and combinations thereof;
wherein the composition does not comprise ethylhexyl-2-cyano-3,3-diphenyl-acrylate (INCI octocrylene).

17. The sunscreen or daily care composition according to claim 16, wherein the composition does not comprise 2-ethylhexyl-(2E)-3-(4-methoxyphenyl)acrylate (INCI ethylhexyl methoxy cinnamate).

18. The sunscreen or daily care composition according to claim 16, wherein the composition is free of parabens.

19. The sunscreen or daily care composition according to claim 16, wherein the at least one organic particulate UV filter is selected from the group consisting of 2,2′-methylene bis[6-(2H-benzotriazol-2-yl)-4-(1,1,3,3-tetramethylbutyl)phenol] (INCI methylene bis-benzotriazolyl tetramethylbutylphenol), 2,4,6-tris(biphenyl-4-yl)-1,3,5-triazine (INCI trisbiphenyl triazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine), 5,6,5′,6′-tetraphenyl-3-3′-(1,4-phenylene)bis(1,2,4-triazine) (INCI phenylene bis-diphenyltriazine), micronized 1,4-di(benzoxazole-2′-yl)benzene and combinations thereof.

20. The sunscreen or daily care composition according to claim 16, wherein the composition further comprises at least one emollient with a polarity index of <30 mN/m.

21. The sunscreen or daily care composition according to claim 20, wherein the at least one emollient is selected from the group consisting of C₁₂-C₁₅ alkyl benzoate, caprylic/capric triglyceride, butylene glycol dicaprylate/dicaprate, propylene glycol dicaprylate/dicaprate, diisopropyl sebacate, octyldodecanol, isopropyl palmitate, isopropyl myristate, dicaprylyl carbonate, phenethyl benzoate, dibutyl adipate, diisopropyl adipate, triethyl citrate and tributyl citrate.

22. The sunscreen or daily care composition according to claim 16, wherein the composition comprises hexyl 2-[4-(diethylamino)-2-hydroxybenzoyl]benzoate (INCI diethylamino hydroxybenzoyl hexyl benzoate) in an amount of from 3% to 8% by weight, based on the total weight of the composition.

23. The sunscreen or daily care composition according to claim 16, wherein the composition comprises the at least one organic particulate UV filter in an amount of from 0.5% to 10% by weight, based on the total weight of the composition.

24. The sunscreen or daily care composition according to claim 16, wherein the composition comprises the at least one UV filter selected from triazine derivatives in an amount of from 0.5% to 5% by weight, based on the total weight of the composition.

25. The sunscreen or daily care composition according to claim 16, wherein the composition comprises at least one perfume.

26. The sunscreen or daily care composition according to claim 16, wherein the composition is free of phenoxyethanol.

27. The sunscreen or daily care composition according to claim 16, wherein the composition has an UV-A protection, which is at least one third of the sun protection factor of the sunscreen composition.

28. A sunscreen or daily care composition, in the form of an emulsion, comprising
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives;
wherein the composition does not comprise ethylhexyl-2-cyano-3,3-diphenyl-acrylate (INCI octocrylene) to reduce fabric staining of a sunscreen or daily care composition.

29. A sunscreen or daily care composition, in the form of an emulsion, comprising
(i) hexyl 2-[4-(diethylamino)-2-hydroxybenzoyl]benzoate (INCI diethylamino hydroxybenzoyl hexyl benzoate); and
(ii) at least one organic particulate UV filter; and
(iii) at least one UV filter selected from triazine derivatives;
wherein the composition does not comprise ethylhexyl-2-cyano-3,3-diphenyl-acrylate (INCI octocrylene) to facilitate the washability of a sunscreen or daily care composition.

30. A sunscreen composition comprising
(i) diethylamino hydroxybenzoyl hexyl benzoate; and
(ii) bis-ethylhexyloxyphenol methoxyphenyl triazine; and
(iii) ethylhexyl triazone; and
(iv) methylene bis-benzotriazolyl tetramethylbutylphenol or trisbiphenyl triazine,
wherein the sunscreen composition has an in vitro Boots rating UVA:UVB ratio of at least 0.9 before and 0.86 after an UV irradiation of 17.5 J/cm².
